(19) United States
(12) Patent Application Publication
(10) Pub. No.: US 2008/0318992 A1
(43) Pub. Date: Dec. 25, 2008

(54) METHOD OF USING A PKC INHIBITOR TO REVERSE PREFRONTAL CORTICAL DECLINES

(75) Inventors: Amy F. T. Arnsten, Bethany, CT (US); Avis Brennan Hains, New Haven, CT (US)

Correspondence Address: CANTOR COLBURN, LLP, 20 Church Street, 22nd Floor, Hartford, CT, 06103, US

(73) Assignee: YALE UNIVERSITY, New Haven, CT (US)

(21) Appl. No.: 12/053,920

(22) Filed: Mar. 24, 2008

Related U.S. Application Data
(60) Provisional application No. 60/919,456, filed on Mar. 22, 2007.

(51) Int. Cl. A61K 31/4355; A61P 25/28

(52) U.S. Cl. 514/280

(57) ABSTRACT

Aging is associated with deficiencies in the prefrontal cortex, including working memory impairment, and compromised integrity of neuronal dendrites. The role of Protein Kinase C(PKC) in age-related prefrontal cortical impairments had not been previously determined. The inventors provides evidence that PKC activity is associated with prefrontal cortical dysfunction in aging. The inventors provide methods of reversing the effects of prefrontal cortical decline, improving cognition, and improving working memory in aged subjects by providing a phenanthridinium alkaloid or closely related compound to the subject. The inventors found chelerythrine reversed working memory impairments in aged rats and enhanced working memory in aged rhesus monkeys. Improvement correlated with age, with older monkeys demonstrating a greater degree of improvement following PKC inhibition. The inventors findings indicate that PKC is dysregulated in aged subjects and that PKC inhibitors may be used to treatment improve cognition in the elderly.

### CROSS REFERENCE TO RELATED APPLICATION

This application claims priority from U.S. Provisional Application No. 60/919,456 filed Mar. 22, 2007, which is hereby incorporated by reference in its entirety.

### STATEMENT OF FEDERALLY FUNDED RESEARCH

The research was supported by NIH grant R37AG06036 to Amy F. T. Arnsten, the Claude D. Pepper Older Americans Independence Center, Yale University School of Medicine (P30 AG21342 N11/NIA) to Amy F. T. Arnsten, and by NSF 2005022484 Graduate Research Fellowship Grant and American Foundation of Aging Research Graduate Research Fellowship (AFAR-GRF) titled “Intracellular Signaling in the Aged Prefrontal Cortex and Behavioral Measures of Prefrontal Integrity” to Avis Brennan Hains. The U.S. government has certain rights in this invention.

### FIELD OF THE INVENTION

The invention includes methods of preventing and reversing prefrontal cortical decline in normal aged subjects by providing a protein kinase C inhibitor to the subject. Compounds, dosage forms, and methods of administration are provided herein.

### BACKGROUND

Studies of aged animals and humans show prominent deficits in prefrontal cortical cognitive functions including working memory (Albert, M. S., Phil. Trans. R. Soc. London (1997) 352: 1703-1709; Ando, S. and Ohashi, Y., Neurosci. Lett. (1991) 128: 17-20; Bartus, F. T. et al., J. Gerontol. (1978) 33: 858-871; Bimonte et al., Neurobiol. Aging (2003) 24: 37-48; Moore, T. L., et al., Behav. Brain Res. (2005) 160: 208-221, Moore, T. L., et al., Neurobiol. Aging (2006) 10: 1484-1493; Nielsen-Bohlman, L. and Knight, R. T., Cereb. Cortex (1995) δ: 541-549; Rapp, P. R. and Amaral, D. G. J. Neurosci. (1989) 3: 568-576; Schacter, D. L., et al., and Neuroreport (1996) 7: 1165-1169). Elucidating mechanisms underlying prefrontal cortical decline is critical for the treatment of debilitating age-related cognitive deficits. Goldman-Rakic (Neuron (1995) 14: 477-485) described working memory microcircuits as local networks, comprised of 1) pyramidal cells engaged in recurrent excitation, creating persistent firing, and 2) GABAergic interneurons, which provide spatial tuning. Prefrontal cognitive impairments may stem from disruptions to the structural components of such microcircuits (Liston, et al., J. Neurosci. (2006) 26: 7870-7874), and from chemical imbalances in prefrontal cortex, e.g. as described below.

Studies of humans and animals have established that advanced aging is associated with reduced dendritic arborizations and spine density (Cupp, C. J. and Uemura, E., Exp. Neurol. (1980) 69: 143-163; Duan, H. et al., Cereb. Cortex (2003) 13: 950-961; Harmon, K. M. and Wellman, C. L. Brain Res. (2003) 992: 60-68; Jacobs, et al., J. Comp. Neurol. (1997) 386: 661-680; Nakamura, et al., Acta Neuropathol. (1985) 65: 281-284; Page, et al., Neurosci. Lett. (2002) 317: 37-41; Peters, A. et al., Cereb. Cortex (1998) 8: 671-684; Scheibel, M. E. et al., Exp. Neurol. (1975) 47: 392-403; Uemura, E., Exp. Neurol. (1980) 69-164-172) of prefrontal cortical pyramidal cells as well as 40-50% reduction in synapse density in superficial layers of the prefrontal cortex (Peters et al., 1998; Peters, A., et al. Cereb. Cortex (2001) 11: 93-103). Thus, the anatomical substrates of prefrontal networks are afflicted in the aged. These changes are likely reflected in the loss of prefrontal gray matter, which has been reported in aged humans (Gunning-Dixon, F. M. and Raz, N., Neuropsychologia (2003) 41: 1929-1041; Raz, N., et al., Cereb. Cortex (1997) 7: 268-282).

It has been shown that protein kinase C(PKC), which comprises a family of kinases that are activated indirectly by Gq-protein-coupled receptor stimulation, is a potent regulator of prefrontal cortical cognitive function (Birnbaum, S. G., et al., Science (2004) 306: 882-884; Runyan, J. D., et al., Learn. Mem. (2005) 12: 103-110). Overactivation of PKC impairs delay-related firing and performance on tasks of working memory via α-1 noradrenergic receptor activation (Birnbaum et al., 2004). Furthermore, PKC activity regulates the actin cytoskeleton (Larsson, C., Cell Signal (2006) 18: 276-284) and overactivation of PKC results in spine loss and altered spine morphology in vitro (Calabrese, B. and Halpain, S. Neuron (2005) 48: 77-90) suggesting a role for PKC overactivation in age-related prefrontal cognitive and structural deficits. Although no studies had previously examined PKC activation within the aged prefrontal cortex, studies of the hippocampus (Colombo, P. J. and Gallagher, M., Hippocampus (2002) 12: 285-289; Colombo, P. J., et al., Proc. Natl. Acad. Sci. (1997) 94: 14195-14199) and the entire cortex (Battaini, F., et al., Neurobiol. Aging (1990), 11: 563-566; Battaini, F., et al., Neurobiol. Aging (1995) 16: 137-148; Battaini, F. and Pascale, A., Ann. NY Acad. Sci. (2005) 1057: 177-192) indicate that the cellular distribution and quantity of activated PKC is altered in the aged brain.

There exists a need to clarify the relationship between activation of PKC and structural and functional integrity of the aged prefrontal cortex. We hypothesized that age-related cognitive impairments involve dysregulation of PKC in the prefrontal cortex. Our findings indicate that working memory performance is inversely related to the activity of PKC in the aged prefrontal cortex, and that inhibition of PKC activity may be useful in the treatment of prefrontal impairments in the elderly.

### SUMMARY

The invention includes methods of preventing and reversing prefrontal cortical decline in normal aged subjects by providing a protein kinase C inhibitor to the subject. The protein kinase C inhibitor can be a phenanthridinium alkaloids or other closely related compound. The inventors have found that when such compounds are administered to normal aged subjects the cognitive performance, including including performance in tasks that utilizing working memory, of the subjects improves.

The invention includes a method for improving cognition, including working memory, in an aged subject comprising providing to the subject an effective amount of a compound or salt of the structure:

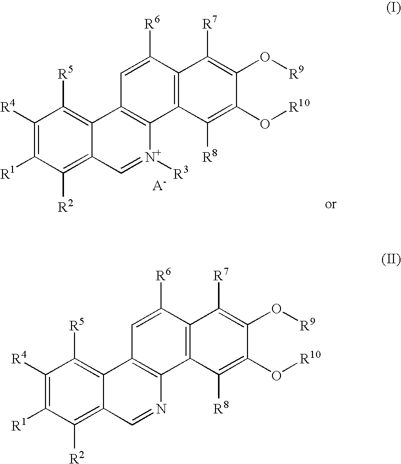

Within Formula (I) and (II) R¹ and R² are independently selected from hydrogen, halogen, C₁-C₃alkyl, C₁-C₃alkoxy, or C₁-C₃alkylester;_R³ is hydrogen or C₁-C₆alkyl; R⁴, R⁵, R⁶, R⁷ and R⁸ are independently selected from hydrogen, hydroxyl, C₁-C₃alkyl, —(CH₂)ₙO(C₁-C₃alkyl), —(CH₂)ₙO—C(═O)—(C₁-C₂)alkyl or —(CH₂)ₙC(═O)—O—(C₁-C₃)alkyl; R⁹ and R¹⁰ are independently H, C₁-C₆ alkyl or R⁹ and R¹⁰ are taken together form a —(CH₂)ₘ— group to produce a 5-7 membered ring; n is from 0 to 3; m is from 1 to 3; and A⁻ is an anion of a pharmaceutical salt, which forms a pharmaceutically acceptable salt with the quaternized amine group.

In certain embodiments the compound used to improve cognition is chelerythrine. The invention pertains to methods of improving cognition in aged human and aged non-human subjects by providing a compound of Formula (I) or (II) to the subject.

The invention includes methods of improving cognition in aged subjects by administering a compound of Formula (I) or (II) non-centrally. For example, the compound of Formula (I) or (II) may be administered orally.

The invention includes methods of improving cognition in an aged human subject in which the method includes informing the subject that the compound of Formula (I) or (II) can be used to improve cognition.

### BRIEF DESCRIPTION OF THE DRAWINGS

FIG. 1. PKC inhibition with CHEL improved working memory performance in aged rhesus monkeys (F_{(3, 36)}=5.654, p=0.003). Vehicle scores did not significantly differ from 3.0 μg/kg or 30.0 μg/kg of CHEL A. Oral administration of 0.3 μg/kg CHEL significantly improved delayed response performance in monkeys (N=13) compared to vehicle (p=0.013, B). Pearson's tests revealed a significant positive correlation between age of the monkey (years) and cognitive improvement with 0.3 μg/kg CHEL (r=0.777, p=0.008, C). There was not a significant relationship between age of the monkey and performance following vehicle treatment (r=0.044, D). All monkeys were maintained at a baseline of 63-70% correct prior to treatment administration (gray bar). Values represent means±SEM. (*) p<0.05.

FIG. 2. PKC inhibition with CHEL rescued working memory impairment in aged rats (F_{(2, 12)}=3.98, p=0.047). Subcutaneous administration of 1.0 mg/kg CHEL significantly improved performance compared to vehicle (p=0.047). Performance following 0.3 mg/kg CHEL was not significantly different from vehicle. Performance following treatment is expressed as change in percent-correct between mean 2-day baseline, and percent-correct following treatment. Values represent means±SEM. (*) p<0.05.

FIG. 3. Aged rats included in correlative studies were cognitively unimpaired. Aged (N=9) and young adult (N=6) rats were trained and characterized for 20 test sessions on a working memory task. Aged rats took significantly longer to habituate than young adult rats (p=0.044, A). Mean percent-correct over the 20 test sessions and for the last 3 test sessions was not significantly different between age groups (B). Values represent means±SEM. (*) p<0.05.

FIG. 4. Prefrontal PKC activation predicted working memory impairment in aged rats. PKC activation was determined by evaluating PKC concentration in the membrane fraction of the medial prefrontal cortex of cognitively characterized rats (FIG. 3) using ELISA. Values were normalized to mean PKC concentration for young adult rats (N=6). Aged (N=9) and young adult rats did not differ in basal PKC activity (A). PKC activation inversely correlated with mean performance over the last 3 test sessions within aged rats (r=−0.780, p=0.013, B, also see FIG. 3B). Working memory performance did not correlate with PKC activation in young adult rats (r=−0.15, C). Values represent means±SEM.

FIG. 5. The effects of chelerythrine on spatial working memory in aged monkeys. The effects of chelerythrine on spatial working memory performance were determined 4 hours after oral administration to aged rhesus monkeys. The 0.3 ug/kg dose improved performance 4 hrs after administration (p=0.012 compared to vehicle), similar to its effects at 1 hr after administration (see FIG. 1). For all monkeys, a best dose could be found that reliably improved performance (p=0.001). Please note that no serious deficits were found, even at high doses (150 ug/kg)

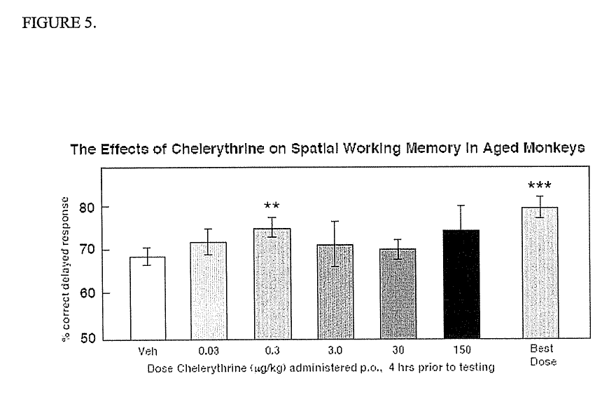

### DETAILED DESCRIPTION

Prior to setting forth the invention in detail, it may be helpful to provide definitions of certain terms and abbreviation to be used herein. Compounds and methods of the present invention are described using standard nomenclature. Unless defined otherwise, all technical and scientific terms used herein have the same meaning as is commonly understood by one of skill in the art to which this invention belongs.

### Abbreviations

CANTAB Cambridge Neuropsychological Test Automated Battery

CHEL Chelerythrine

PKC Protein Kinase C

SEM Standard error of the mean

Terminology

The terms “a” and “an” do not denote a limitation of quantity, but rather denote the presence of at least one of the referenced item. The term “or” means “and/or”. The terms “comprising”, “having”, “including”, and “containing” are to be construed as open-ended terms (i.e., meaning “including, but not limited to”). Recitation of ranges of values are merely intended to serve as a shorthand method of referring individually to each separate value falling within the range, unless otherwise indicated herein, and each separate value is incorporated into the specification as if it were individually recited herein. The endpoints of all ranges are included within the range and independently combinable. All methods described herein can be performed in a suitable order unless otherwise indicated herein or otherwise clearly contradicted by context. The use of any and all examples, or exemplary language (e.g., “such as”), is intended merely to better illustrate the invention and does not pose a limitation on the scope of the invention unless otherwise claimed. No language in the specification should be construed as indicating any non-claimed element as essential to the practice of the invention as used herein. Unless defined otherwise, technical and scientific terms used herein have the same meaning as is commonly understood by one of skill in the art to which this invention belongs.

An “active agent” means a compound (including a compound of the invention), element, or mixture that when administered to a subject, alone or in combination with another compound, element, or mixture, confers, directly or indirectly, a physiological effect on the patient. The indirect physiological effect may occur via a metabolite or other indirect mechanism. When the active agent is a compound, then salts, solvates (including hydrates) of the free compound, crystalline forms, non-crystalline forms, and any polymorphs of the compound are included.

In certain situations, the compounds of formula (I) and (II) may contain one or more asymmetric elements such as stereogenic centers, stereogenic axes and the like, e.g. asymmetric carbon atoms, so that the compounds can exist in different stereoisomeric forms. These compounds can be, for example, racemates or optically active forms. For compounds with two or more asymmetric elements, these compounds can additionally be mixtures of diastereomers. For compounds having asymmetric centers, it should be understood that all of the optical isomers and mixtures thereof are encompassed. In addition, compounds with carbon-carbon double bonds may occur in Z- and E-forms, with all isomeric forms of the compounds being included in the present invention. In these situations, the single enantiomers, i.e., optically active forms, can be obtained by asymmetric synthesis, synthesis from optically pure precursors, or by resolution of the racemates. Resolution of the racemates can also be accomplished, for example, by conventional methods such as crystallization in the presence of a resolving agent, or chromatography, using, for example a chiral HPLC column.

Where a compound exists in various tautomeric forms, the invention is not limited to any one of the specific tautomers, but rather includes all tautomeric forms.

“Compounds of Formula (I) and (II)” includes prodrugs of Formula (I) and (II) unless otherwise stated or clearly contraindicated by the context. Certain prodrugs of Formula (I) and (II) are described in detail below.

The present invention is intended to include all isotopes of atoms occurring in the compounds described herein. Isotopes include those atoms having the same atomic number but different mass numbers. By way of general example, and without limitation, isotopes of hydrogen include tritium and deuterium and isotopes of carbon include ¹¹C, ¹³C, and ¹⁴C.

“Pharmaceutically acceptable salts” include derivatives of the disclosed compounds wherein the parent compound is modified by making non-toxic acid or base salt thereof, and further refers to pharmaceutically acceptable solvates of such compounds and such salts. Examples of pharmaceutically acceptable salts include, but are not limited to, mineral or organic acid salts of basic residues such as amines; alkali or organic salts of acidic residues such as carboxylic acids; and the like. The pharmaceutically acceptable salts include the conventional non-toxic salts and the quaternary ammonium salts of the parent compound formed, for example, from non-toxic inorganic or organic acids. For example, conventional non-toxic acid salts include those derived from inorganic acids such as hydrochloric, hydrobromic, sulfuric, sulfamic, phosphoric, nitric and the like; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, mesylic, esylic, besylic, sulfanilic, 2-acetoxybenzoic, fumaric, toluenesulfonic, methanesulfonic, ethane disulfonic, oxalic, isethionic, HOOC—(CH₂)ₙ—COOH where n is 0-4, and the like. The pharmaceutically acceptable salts of the present invention can be synthesized from a parent compound, a basic or acidic moiety, by conventional chemical methods. Generally, such salts can be prepared by reacting free acid forms of these compounds with a stoichiometric amount of the appropriate base (such as Na, Ca, Mg, or K hydroxide, carbonate, bicarbonate, or the like), or by reacting free base forms of these compounds with a stoichiometric amount of the appropriate acid. Such reactions are typically carried out in water or in an organic solvent, or in a mixture of the two. Generally, non-aqueous media like ether, ethyl acetate, ethanol, isopropanol, or acetonitrile are preferred, where practicable. Lists of additional suitable salts may be found, e.g., in Remington's Pharmaceutical Sciences, 17th ed., Mack Publishing Company, Easton, Pa., p. 1418 (1985).

Certain compounds are described herein using a general formula that includes variables, e.g. R, R₁, R₂, and R₃. Unless otherwise specified, each variable defined independently of each other variable in the formula. Thus, if a group is said to be substituted, e.g. with 0-2 R*, then said group may be substituted with up to two R* groups and R* at each occurrence is selected independently. Also, combinations of substituents and/or variables are permissible only if such combinations result in stable compounds. A stable compound is a compound sufficiently robust to survive isolation from a reaction mixture and subsequent formulation into an effective therapeutic agent.

The term “substituted”, as used herein, means that any one or more hydrogens on the designated atom or group is replaced with a selection from the indicated group, provided that the designated atom's normal valence is not exceeded. When the substituent is oxo (i.e., ═O), then 2 hydrogens on the atom are replaced. When aromatic moieties are substituted by an oxo group, the aromatic ring is replaced by the corresponding partially unsaturated ring. For example a pyridyl group substituted by oxo is a pyridone. Combinations of substituents and/or variables are permissible only if such combinations result in stable compounds or useful synthetic intermediates.

A dash (“-”) that is not between two letters or symbols is used to indicate a point of attachment for a substituent. For example, —(CH₂)C₃-C₈cycloalkyl is attached through carbon of the methylene (CH₂) group.

“Alkyl” means both branched and straight chain saturated aliphatic hydrocarbon groups, having the specified number of carbon atoms, generally from 1 to about 8 carbon atoms. When C₀-Cₙ alkyl is used herein in conjunction with another group, for example, (carbocycle)C₀-C₄ alkyl, the indicated group, in this case a carbocycle, is either directly bound by a single covalent bond (C₀), or attached by an alkyl chain having the specified number of carbon atoms, in this case from 1 to about 4 carbon atoms. Examples of alkyl include, but are not limited to, methyl, ethyl, n-propyl, isopropyl, n-butyl, 3-methylbutyl, t-butyl, n-pentyl, and sec-pentyl. Preferred alkyl groups are those having from 1 to about 6 carbon atoms, or from 1 to about 4 carbons atoms e.g. C₁-C₆ and C₁-C₄ alkyl groups.

“Alkylester” is an alkyl group as defined above attached through an ester linkage. The ester linkage may be in either orientation, e.g. a group of the formula —O(C═O)alkyl or a group of the formula —(C═O)Oalkyl.

The term “effective amount” of a compound of this invention means an amount effective, when administered to an aged subject, to provide a therapeutic benefit such as an amelioration of symptoms or improvement in status, e.g., an amount effective to decrease the symptoms of a disorder or disease or improve function. In certain embodiments an effective amount is an amount sufficient to reduce or reverse prefrontal cortical decline, improve cognitive function in an aged subject, or improve working memory in an aged subject. In certain circumstances the aged subject may not present symptoms of being affected. Thus a therapeutically effective amount of a compound is also an amount sufficient to prevent a significant prefrontal cortical decline, including to prevent a decrease in cognitive function and to prevent a decrease in working memory. An improvement in cognitive function or working memory is any detectable positive change that is statistically significant in a standard parametric test of statistical significance such as Student's T-test, where p<0.

“Informing” means referring to or providing, published material, for example, providing an active agent with published material to a user; or presenting information orally, for example, by presentation at a seminar, conference, or other educational presentation, by conversation between a pharmaceutical sales representative and a medical care worker, or by conversation between a medical care worker and a patient; or demonstrating the intended information to a user for the purpose of comprehension.

“Providing” means giving, administering, selling, distributing, transferring (for profit or not), manufacturing, compounding, or dispensing.

“Treatment,” as used herein includes providing a compound of Formula (I) or (II) sufficient to: (a) prevent prefrontal cortical decline in an aged subject (b) inhibiting the prefrontal cortical decline in an aged subject, i.e. arresting its development; and (c) reversing the effects of prefrontal cortical decline, i.e., causing improvement in prefrontal cortical function.

### Compounds

Compounds useful in the invention include phenanthridinium alkaloids and related compounds of Formula I and II.

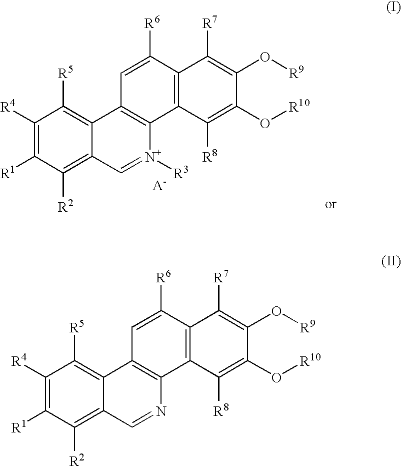

In addition to the definitions given the variables R¹ to R¹⁰ in the “Summary of Invention” these variables may carry any of the definitions set forth below. Definitions of variables R¹ to R¹⁰ may be present in any combination that results in a stable compound of Formula (I) or (II).

R¹ and R² are independently selected from hydrogen, halogen, C₁-C₃alkyl and C₁-C₃alkoxy.

R¹ and R² are both OCH₃ groups.

R³ is H or a C₁-C₂ alkyl group.

R³ is a CH₃ group.

Any or all of R⁴, R⁵, R⁶, R⁷ and R⁸ are each hydrogen.

All of R⁵, R⁶, and R⁷ are hydrogen.

R⁹ and R¹⁰ are each independently H or CH₃.

R⁹ and R¹⁰ are taken together to form a five-membered ring.

is Cl—, citrate, sulfate, or phosphate.

In certain embodiments the compound is chelerythrine or a pharmaceutically acceptable salt thereof.

In some circumstances the compounds may be a pseudobase of a phenanthridium alkaloid. Such pseudobases have the general structure:

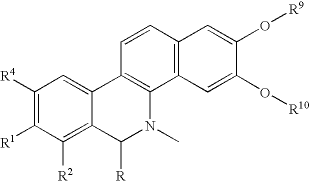

where R¹, R², R⁴, R⁹, and R¹⁰ carry any of the definitions set forth for compounds of Formula (I) and (II) and R is a metabolically labile group such as an alkoxy, alkylamino, alkylcarboxamide, sulfonyl, or alkylester group. For example R may be any of the following:

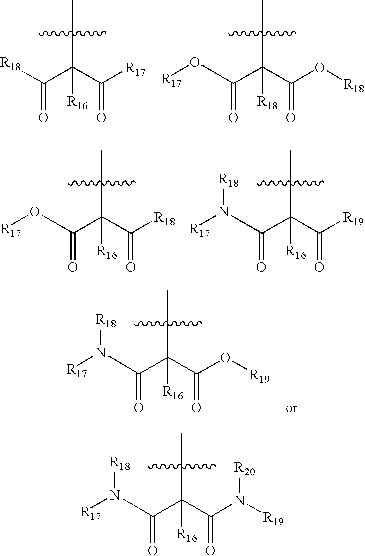

where each of R16, R17, R18, R19, and R20 are independently hydrogen, or C1-C8alkyl, C2-C4alkanoyl, or (C3-C7cycloalkyl)C0-C2alkyl, each of which is substituted with from 0 to 2 substituents independently chosen from halogen, hydroxyl, amino, cyano, C1-C4alkyl, C1-C4alkoxy, mono- and di-(C1-C4allyl)amino, C1-C2haloalkyl, and C1-C2haloalkoxy. Such compounds have been described previously for treating certain disorders of the prefrontal cortex in U.S. Provisional application 60/834,375, filed Jul. 31, 2006, from which published PCT application WO 2008/016596 claims priority. U.S. Provisional application Ser. No. 60/834,375 is hereby incorporated by reference for its teachings regarding pseudobase benzo[c]phenanthridine compounds.

Effect of Chelerythrine on Cognition in Aged Subject

Chelerythrine (CHEL, LC Laboratories, Woburn, Mass.) is known to inhibit PKC activation by blocking the site of diacylglycerol/phorbol ester binding and by inhibiting PKC translocation to the membrane for activation. Systemic administration of CHEL has previously been shown to reverse working memory impairments following administration of the α-1 noradrenergic receptor antagonist, cirazoline, which activates PKC via the Gq signaling cascade (Birnbaum, et al., 2004).

Results provided in the EXAMPLES section indicate that PKC activation is associated with cognitive and morphological deficits of the aged prefrontal cortex. PKC activation within the medial prefrontal cortex predicted poorer working memory performance, and decreased basal dendritic material in aged rats. Inhibition of PKC with systemic administration of CHEL has been shown to improve cognitive performance, including working memory performance in aged, cognitively impaired subjects. The effect of CHEL on cognitive performance in aged subjects providing further evidence that PKC dysregulation contributes to age-related prefrontal deficits.

Low to moderate doses of systemically administered CHEL improved working memory performance in aging rhesus monkeys. Higher does of CHEL produced no improvement. PKC serves a multitude of regulatory functions throughout the brain (Mathis, C., et al., Eur. J. Neurosci. (1992)220: 107-110; Paylor, R., et al., (1991) Behav. Brain Res. 45:189-193; Serrano, P. A. et al., Behav. Neural. Biol. (1994) 61: 60-72; Yang, H. C. and Lee, E. H., Chinese J. Physiol. (1993) 36:115-123; Zhao, W. Q., et al., Behav. Brain Res. (1994) 60: 151-160). The relatively narrow therapeutic range for CHEL may be due to disruptions in homeostatic mechanisms that offset improving effects of CHEL at higher doses. The correlation of age of the monkey with performance on the working memory task following an optimal dose of CHEL suggests that improved cognitive performance following CHEL administration is due to improvements in PKC regulation in older monkeys.

This effect is not due to ceiling effects as all monkeys were held at a 2-day baseline of 60-73% correct. Instead, these findings indicate that PKC is implicated in prefrontal cognitive operations in the aged, which is consistent with previous findings of PKC dysregulation in the aged brain (Battaini et al., 1990, 1995; Battaini and Pascale, 2005; Colombo, P. J. and Gallagher, M., Hippocampus (2002) 12: 285-289; Colombo, P. J., et al., Proc. Natl. Acad. Sci. USA (1997) 94: 14195-14199. In support of this, we found that the oldest 3 monkeys performed >20% better than the 3 youngest monkeys following administration of 0.3 μg/kg CHEL.

In order to determine whether PKC inhibition can rescue age-related impairments in working memory in rats, we examined the effects of acute CHEL on spatial delayed alternation performance in aged rats with mean pretreatment baselines below 60% correct. CHEL significantly improved performance, suggesting that PKC activation contributes to age-related cognitive impairments. Although CHEL was administered systemically, it is likely that the beneficial actions of CHEL arise in the prefrontal cortex. We have observed that systemic CHEL administration and infusions of CHEL directly to the rat medial prefrontal cortex via an implanted cannula are both capable of reversing stress induced working memory impairments (Birnbaum et al., 2004) suggesting that the location of action of systemic CHEL is the prefrontal cortex.

We next examined whether basal PKC activity in the prefrontal cortex is associated with working memory performance in aged and young adult rats. Consistent with our findings of CHEL enhancing working memory function in aged rats and monkeys, basal PKC activation within the prefrontal cortex predicted poorer working memory performance in aged but not young adult rats. Working memory is often impaired in aged animals and humans (Albert, 1997; Ando and Ohashi, 1991; Bartus et al., 1978; Bimonte et al., 2003; Moore et al., 2005, 2006; Nielsen-Bohlman and Knight, 1995; Rapp and Amaral, 1989; Schacter et al., 1996; West, R. L., Psychol. Bull. (1996) 120: 272-292) but deficits can be highly variable, with some aged subjects performing as well as young subjects (Arnaiz et al., 2004; Gage, F. H., et al., Neurobiol. Aging (1989) 10:147-152; Olton et al., 1991; West et al., 2002). In our study, variations in PKC activation were robust enough to explain relatively subtle differences in performance amongst cognitively intact aged rats. These findings are consistent with previous reports of PKC-dependent working memory impairments (Birnbaum et al., 2004; Runyan, J. D., et al., Learn. Mem. (2005) 12: 103-110) and emphasize the role of PKC in age-related working memory function.

Interestingly, there were no age-related differences in basal levels of cytosolic or membrane-bound PKC. Together with our findings of a significant correlation of PKC activity and working memory performance in the aged, this suggests that the effects of PKC are qualitatively different between the young adult and aged prefrontal cortex. Qualitative differences in PKC regulation have been observed in the young vs. aged hippocampus (Colombo and Gallagher, 2002; Colombo et al., 1997). For example, performance on a hippocampus-mediated task was shown to inversely correlate with cytosolic PKCγ in aged rats. Membrane-bound PKCγ (activation) positively correlated with performance in younger rats, however, elevated membrane-bound PKCγ did not predict enhanced performance among the aged (Colombo et al., 1997). The PKC assay used in the present study quantified all membrane-bound PKC isoforms. Thus, suppression or elevation of the activity state of specific isoforms could not be resolved. Elucidating how PKC is (dys)regulated in the aged prefrontal cortex will require these rigorous analyses that incorporate both cellular location-specific and isoform-specific PKC activity states in young adult and aged animals.

Previous attempts to quantify PKC (across isoforms) within the entire cortex of aged rats have produced mixed results. Our findings are in agreement with previous reports of similar cytosolic and membrane-bound PKC concentrations in young vs. aged rat cortex (Battaini et al., 1995), but contrast with earlier reports of reduced membrane PKC in aged Sprague-Dawley rat cortex (Battaini et al., 1990). These discrepancies may reflect variability among aged rats, among isoforms of PKC, or regional differences in PKC regulation. Different cortical areas subserve different functions, thus homogeneity in PKC regulation should not be expected among widespread regions of the cortex. In the present investigation, PKC activation was quantified specifically within the medial prefrontal cortex. Similar membrane and cytosolic concentrations suggest that translocation of PKC to the membrane is not impaired and that the number of functional enzyme molecules is not altered within the aged prefrontal cortex.

A limitation of the present study was the successful performance of the aged rats in the PKC characterization study. There is high variability in the aged population (Amaiz et al., 2004; Gage et al., 1989; Olton et al., 1991; West et al., 2002). This sample of aged rats may have come from a relatively unimpaired population. These cohort effects are a weakness of aging studies, which rely on the limited availability of a very small number of animals at any one time. Despite similar cognitive performance and PKC activation measures, the correlative and pharmacological data indicate that PKC is implicated in prefrontal cognitive function in the aged. Inclusion of a more functionally heterogeneous sample of aged animals for correlative studies would provide a better indication as to how PKC activation-levels in the aged compare to the young adult levels and predict working memory across the spectrum of cognitive abilities. Further experiments are also required to determine if PKC regulatory mechanisms, such as inactivation of PKC, or phorbol 12-myristate 13-acetate (PMA)-induced translocation of PKC from the cytosol to the membrane, are altered within the aged prefrontal cortex.

The working memory functions of the prefrontal cortex depend on the interconnectivity of recurrent microcircuits (Goldman-Rakic, 1995). Pyramidal cells are the principle components of these networks, and dendritic spines are a fundamental anatomical substrate of excitatory network activity. PKC is implicated in neuronal plasticity and is capable of regulating the actin cytoskeleton and therefore the motility of dendritic spines (for review see Larsson, 2006). Dysregulation of PKC in the aged may provide a mechanistic link to findings of decreased dendritic spine density and dendritic remodeling in the aged prefrontal cortex.

In summary, inhibition of PKC rescued age-related working memory impairments in both aged rats and monkeys. In aged rats, PKC activity was inversely associated with cognitive function. These findings indicate that PKC is associated with prefrontal cortical dysfunction in the aged and suggests that PKC inhibitors may be useful to improve cognition in the elderly. Future studies examining age-specific alterations in PKC isoforms or directly manipulating PKC activation within the prefrontal cortex are required to fully elucidate the nature of PKC dysregulation as well as the potential mechanistic role of PKC in age-related prefrontal cognitive and structural decline.

### Methods of Treatment

The invention provides a method of treating a an aged subject comprising administering an effective amount of a compound of Formula (I) or (II) or other related compound to the subject. The subject1 will typically be an aged human patient, but methods of treating domesticated companion animals (pets, such as dogs) are also within the scope of the invention.

Frequency of dosage may vary depending on the compound used and the particular condition to be treated or prevented. In general, a dosage regimen of 4 times daily or less is preferred. For the improving cognition and/or improving working memory in an aged subject a dosage regimen of 1 or 2 times daily is particularly preferred. It will be understood, however, that the specific dose level for any particular patient will depend upon a variety of factors including the activity of the specific compound employed, the age, body weight, general health, sex, diet, time of administration, route of administration, rate of excretion, drug combination and the severity of the particular disease in the subject undergoing therapy. In certain embodiments, administration at meal times is preferred. In general, the use of the minimum dosage that is sufficient to provide effective therapy is preferred. Subjects may generally be monitored for therapeutic effectiveness using assays suitable for the condition being treated or prevented, which will be familiar to those of ordinary skill in the art.

Methods of treatment include methods in which cognition is improved and/or working memory is improved in an aged subject provided a compound of Formula (I) or (II). In certain embodiments the compound is chelerythrine. Methods of treatment also include methods in which an aged subject provided with a compound of Formula (I) or (II), does not experience a decline in prefrontal cortical performance and/or stable working memory performance over time. In some embodiments an effective amount of a compound of Formula (I) or (II) is an amount effective to produce a significant improvement in a standard test of cognitive performance as compared to the subject's performance before administering the compound.

Methods of assessing cognitive function in aged human subjects include any commonly accepted test for assessing cognitive performance in humans. Examples include, but are not limited, to assessments by the spatial working memory task on the CANTAB, the Stroop interference task, the Tower of London planning task, the Wisconsin Card Sort, and measures of recall (but not recognition) memory. In some embodiments an effective amount of a compound of Formula (I) or (II) is an amount effective to produce a significant improvement in the subject in a standard test of cognitive function as compared to the subject's performance in the test of cognitive function before administration of the compound.

Methods of assessing working memory performance in aged human subjects include any commonly accepted test for assessing working memory performance in humans. Typically, these tests assess subjects' ability to remember information in the presence of interference, e.g. from distractors or prepotent habits, and to use this information to plan, organize, and regulate thought and action. In some embodiments an effective amount of a compound of Formula (I) or (II) is an amount effective to produce a significant improvement in the subject in a standard test of working memory as compared to the subject's performance in the test of working memory before administration of the compound.

Onset of age related declines in prefrontal cortical function vary among individuals, however in some embodiments the subject will be a human subject of age 55 or greater or age 70 or greater.

The dose required to prevent or reverse age related prefrontal cortical decline varies according to the subject treated and the particular compound used; in some embodiments from about 0.1 micrograms/kg chelerythrine to about 10 micrograms/kg chelerythrine are administered to the subject per day.

The invention included methods in which the compound in administered non-centrally. The invention includes embodiments in which the compound is administered orally.

The compound will typically be administered as a unit dosage form. The dosage form may be any pharmaceutically acceptable dosage form. Dosage formulations suitable for oral use, include, tablets, troches, lozenges, aqueous and oily suspensions, dispersible powders or granules, emulsions, hard or soft capsules, and syrups and elixirs. Dosage forms also include injectable, parenteral, topical, and transdermal dosage forms. In certain embodiments the aged subject is provided a unit dosage form containing from about 3 micrograms to about 300 micrograms of the compound. In certain embodiments the compound contained in the dosage form is chelerythrine.

Methods of treatment include methods in which the compound of Formula (I) or (II) is the only active agent administered to the subject or in which the compound of Formula (I) or (II) is given together with one or more other active agents.

Methods of treatment include methods of informing the aged subject that a compound of Formula (I) or (II) can be used improve cognition and/or working memory in aged subjects. The informing may be by any means of providing information including product labeling.

### EXAMPLES

### Example 1

### Working Memory Performance and PKC Inhibition in Non-Human Primates

Performance on this task is reliant upon the integrity of the dorsolateral prefrontal cortex (Goldman-Rakic, 1987) and is consistently impaired in aged rhesus monkey (Bartus, et al., 1978; Rapp and Amaral, 1989).

The present study used 13 aged female rhesus monkeys (Macaca mulatta) ranging in age from 18 to 35 years at the time of testing. Actual birthdates were not available for several of the wild-caught animals and ages were estimated based on health records, teeth, and known history; several had been in the Yale University colony for more than 15 years. Monkeys were individually housed and maintained on a diet of Purina monkey chow (St. Louis, Mo.) supplemented with fruit. All procedures were conducted at Yale University, and were approved by the Yale Animal Care and Use Committee and were in accordance with the National Institute of Health's Guide for Care and Use of Laboratory Animals.

Animals were tested at the same time of day immediately prior to feeding. Highly palatable food rewards (e.g., peanuts, raisins, or chocolate chips) were utilized during testing to minimize the need for dietary regulation. Animals were tested twice a week with 3-4 days separating each test session (e.g., Monday and Thursday). Cognitive testing of monkeys occurred in a Wisconsin General Testing Apparatus (WGTA) situated in a sound-attenuating room. Background masking noise (60 dB, wideband) was used to minimize auditory distractions.

The monkeys were trained on the spatial delayed response task as described previously (Arnsten, A. F., et al., J. Neurosci. (1988) δ: 4287-4298). For the delayed response task, the animal watched as the experimenter baited one of several food-wells with a food reward. The number of food-wells varied from two to four wells, depending on the monkey's performance level and experience with the task. Care was taken by the experimenter to ensure that the animal attended the baiting procedure. After baiting, food-wells were covered with identical plaques, and an opaque screen was lowered between the animal and the food-wells for a specified delay. At the end of the delay, the screen was raised and the animal was allowed to choose. Reward was quasi-randomly distributed between the wells. Five delay lengths (referred to as delays A through E) were quasi-randomly distributed over 30 trials. The shortest of these delays was ˜2 s (the “0” s, A delay). The remaining delays were titrated for individual monkeys such that each monkey maintained a baseline performance of 60-73% correct. For example, the delays for one animal might be A=0, B=5, C=10, D=15, and E=20 s.

Data were analyzed by comparing performance following treatment using a one-way repeated-measures ANOVA. Pair-wise comparisons were evaluated using Fischer's Lest Significant Difference (LSD) test. Values were represented as means±SEM. Linear relationships between treatment response at the optimal dose of CHEL and age of the monkey (years) were analyzed in 10 monkeys with known birthdates using Pearson's test. In all cases, an α level of 0.05 was considered statistically significant.

Monkeys (N=13) were orally administered 0.3 μg/kg, 3.0 μg/kg or 30.0 μg/kg of Chelerythrine (CHEL, LC Laboratories, Woburn, Mass.) dissolved in vehicle (water) 1 h prior to testing by a single experimenter who was unaware of the drug treatment conditions. All animals received all treatments conditions, with at least 7 days between treatments. The order of treatment administration was quasi-randomized.

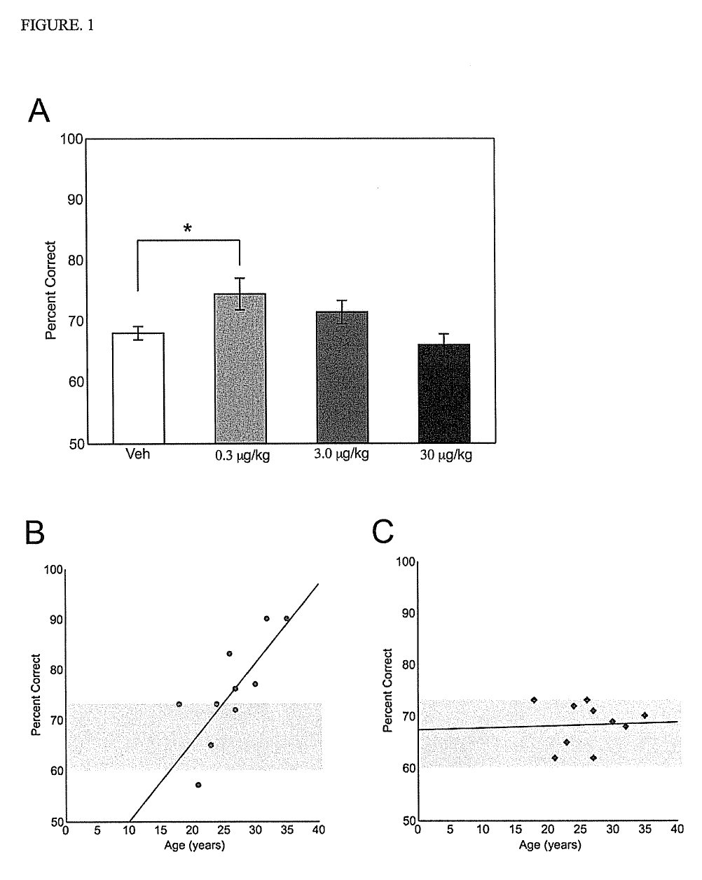

Task difficulty was adjusted to maintain each monkey at a 2-day baseline average of 60-73%. A one-way repeated-measures ANOVA revealed a significant effect of treatment on task performance (F_{(3, 36)}=5.654, p=0.003). Pair-wise comparisons indicated that doses of 0.3 μg/kg CHEL significantly improved performance with respect to vehicle (0.3 μg/kg CHEL: 74.5±2.6% correct, Veh: 68.1±1.1% correct, p=0.013, FIG. 1A). Vehicle scores did not significantly differ from higher doses of CHEL (3.0 μg/kg CHEL: 71.5±1.9% correct, p=0.151; 30.0 μg/kg CHEL: 66.1±1.8%, p=0.406).

Pearson's test revealed a significant correlation between the age of the monkey and cognitive performance following treatment with 0.3 μg/kg CHEL, whereby older age was associated with greater improvement (Pearson's test: r=0.777, p=0.008, FIG. 1B). As delays were adjusted to maintain each monkey at 60-73% correct prior to treatment, there were no ceiling effects in the younger aged monkeys. No correlation of age with cognitive performance was observed following vehicle treatment (Pearson test: r=0.044, p=0.904, FIG. 1C).

Following 0.3 μg/kg CHEL, performance of the 3 oldest monkeys (mean age=32.3 years) was 85.6±4.4% correct, which was >20% better than the performance of the 3 youngest aged monkeys (mean age=20.7 years, 65.0±4.8% correct). Performance following vehicle was comparable across age groups (oldest: 69.0±0.7% correct; youngest: 66.8±3.4% correct, data not shown). Thus PKC inhibition with CHEL had a beneficial influence on a prefrontal mediated cognitive task, and these beneficial effects were more pronounced in the oldest monkeys. This suggests changes in endogenous PKC actions with advancing age.

### Example 2

### Working Memory Evaluation in Rats

Sixteen aged (23.5-25 months) and 6 young adult (3-5 months) male Sprague-Dawley rats from Harlan (Indianapolis, Ind.) were used for two studies. Aged rats arrived at 20 months and were tested between 23.5 and 25 months of age. Young adult rats were tested at 3-5 months of age. Of note, the 16 aged rats arrived in two separate shipments of 6 and 10 rats. We have previously observed variability in performance and health between shipments of aged rats (unpublished observations). To minimize variability, shipments remained separate for the two studies. Unless otherwise noted, rats in both studies were exposed to identical housing, feeding and handling procedures. All rat training, testing and drug administration procedures were performed at Yale University and were approved by the Yale Animal Care and Use Committee and were in accordance with the National Institute of Health's Guide for Care and Use of Laboratory Animals.

Rats were single housed in filter-frame caged and kept on a 12 h light/dark cycle. Experiments were conducted during the light phase. Starting one day after arrival, rats were habituated to a restricted diet (˜13 g/day per rat) of autoclaved Purina (St. Louis, Mo.) rat chow. For the duration of the study, food was administered immediately after rats were behaviorally tested and water was available ad libitum. Rats were weighed weekly to confirm that they were not undergoing any irregular weight loss due to the regulated diet. Food rewards during behavioral testing were highly palatable miniature chocolate chips. Behavioral testing was conducted 5 days per week and was always conducted in the same room and within the same two-hour time frame (9:00 am-11:00 am).

Rats were trained and tested on the spatial delayed alternation task in the T-maze, as described in (Birnbaum, S. et al., Biol. Psychiatry (1999) 46: 1266-1274). This task is reliant upon the medial prefrontal cortex (Divac, I., Neuropsychologia (1971) 9: 175-183) and uses a number of processes associated with prefrontal cortical function including spatial working memory (Kesner, R. P., Exp. Brain Res. (1989) 74: 163-167; Kolb, B. and Gibb, R., Prog. Brain Res., (1990) 85: 241-256). Special care was taken to habituate all rats to a T-maze (dimensions, 90×65 cm). The habituation period was defined as the number of training sessions until the rats were eating 12 chocolate-chip rewards offered at either end of the T-maze from the hand of an experimenter, with 12 pick-ups and replacements to the start-box of the maze between rewards. Habituation and testing criteria were different for the two studies and are described in detail below.

Following habituation, performance was evaluated on the spatial delayed alternation task. On the first trial, rats were rewarded for entering either arm. Thereafter, for a total of 12 trials per session, rats were rewarded only if they entered the maze arm that was not previously chosen. Between trials, the choice point was wiped with alcohol to remove any olfactory clues. Delay periods between trials ranged from 2 s (the minimum possible for delayed alternation, referred to as “0 s”) to 15 s.

### Example 3

### Acute PKC Inhibition in Aged, Cognitively-Impaired Rats

Aged rats (N=6) arrived in a single shipment and were habituated and trained on the spatial working memory task described above. Animals were allowed a maximum time of 20 minutes to complete all 12 trials. Criterion for enrollment in this study was a mean 2-day performance of at least 58% correct at a 0 s delay within 20 min. One rat failed to meet criterion and was eliminated from the study. Rats were tested once per day 5 days per week throughout the duration of the study. Delays were titrated in increments of 5 s to maintain animals at a baseline of 58-75% correct. Rats performing below this baseline subsequent to training were tested at the lowest possible delay (0 s). Delays did not exceed 15 s throughout the study.

Rats were approximately 24 months old upon initiation of pharmacological testing. Each rat was administered each of 3 treatment conditions: 0.3 mg/kg CHEL, 1.0 mg/kg CHEL or vehicle (water) via subcutaneous injection 45 min prior to testing. Care was taken to habituate all rats to injections prior to start of the study. Treatment order was quasi-randomized and treatments were separated by a minimum of 6 days.

Due to variability in performance within the same animal, outcome following treatment was measured as the difference in percent-correct following treatment and mean percent-correct over the 2 days prior to the treatment. Treatment was administered only when prior baseline was stable, which was defined as 2 consecutive scores at the same delay within 20 percentage-points. Comparisons were made using one-way ANOVA and pair-wise comparisons were evaluated using Fischer's LSD test. Values were represented as means±SEM.

The effect of CHEL on working memory was evaluated in a group of aged rats with substantial working memory impairment. Mean baseline performance prior to the first treatment was below 60% correct. All rats included in this study (N=5) were required to demonstrate adequate training by completing the task with above-chance performance on at least 2 consecutive days prior to the first treatment. Approximately 12 weeks of training was required.

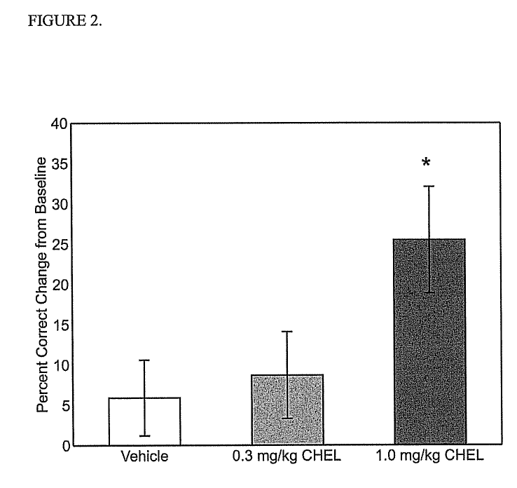

Performance following acute treatment with either CHEL (0.3 mg/kg or 1.0 mg/kg) or vehicle was evaluated by comparing the difference between treatment performance and the mean performance from the previous 2 days. Thus, performance is expressed as percent-change (improvement or impairment from previous baseline). A one-way ANOVA revealed a significant effect of drug treatment (F_{2, 12)}=3.98, p=0.047, FIG. 2). Post hoc analysis using Fischer's LSD test indicated that doses of 1.0 mg/kg CHEL significantly improved performance compared to vehicle (1.0 mg/kg CHEL: 26±6.4% improvement; Veh: 5.4±1 4.7% improvement, p=0.047). Performance following 0.3 mg/kg CHEL was not significantly different from vehicle (0.3 mg/kg CHEL: 8.6±5.4% improvement, p=0.691). PKC inhibition did not appear to influence performance through non-specific motor effects as performance times did not differ as a result of treatment (data not shown). These findings indicate that PKC inhibition attenuates age-related impairments in working memory.

### Example 4

### Cognitive Characterization of Aged, Cognitively-Unimpaired Rats

The relationship between PKC inhibition and working memory performance in the aged suggest that age-related impairments in prefrontal cortical function involve dysregulated PKC activity. To examine this, aged and young adult rats were characterized on the spatial delayed alternation task and PKC activation.

Comparisons in cognitive performance were made using independent t-tests.

Young adult (N=6) and aged (N=10) rats were trained on the spatial working memory task described above. All animals were allowed no more than 10 min to complete 12 trials (habituation and testing). Once habituated, rats were trained to alternate at a short delay (0 s) until they achieved a 2 day average of at least 67% correct. Criteria for training and habituation were developed to minimize the potential confound of aged animals increasing delay times due to slow performance. One aged rat failed to meet criterion and was eliminated from the study. Rats were cognitively characterized for 20 testing sessions, over 30 days. Of note, the 9 aged rats included in this study performed more consistently and at a higher baseline than the 5 aged rats included in the acute PKC inhibition study illustrating the great variability in cognitive status among aged rats.

Rats were tested once per day, 5 days per week on a 12-trial task. Four different delays were quasi-randomized throughout the task. Delays used for this study were: 0, 5, 10 and 15 s. Performance was measured as number of trials correct out of 12 and these percentages were compared between age groups over the entire 20 session characterization period. Performance during the last 3 testing sessions of the characterization period was calculated to reflect performance proximal to the time of sacrifice, when morphological and biochemical measures were obtained (see below). Group means were compared using an independent t-test and values were represented as the mean±SEM.

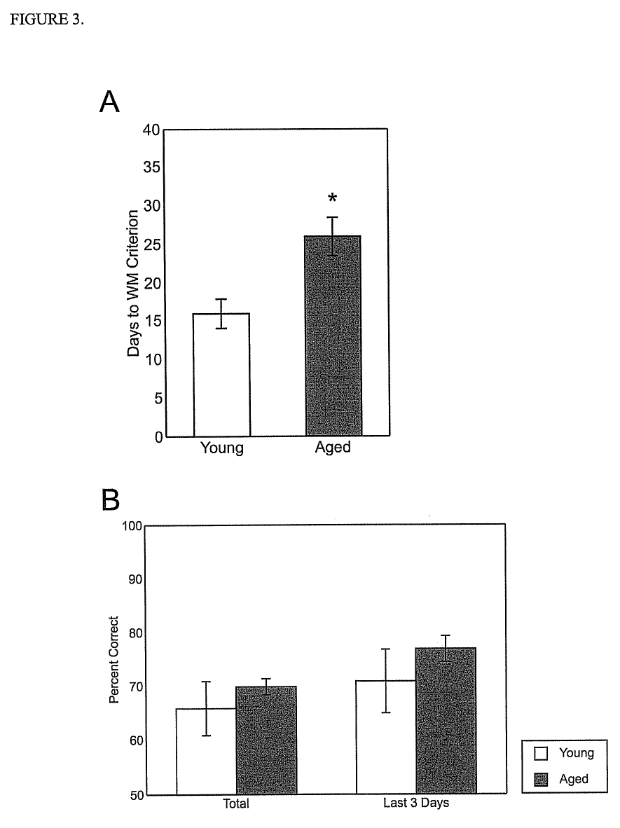

Aged rats took significantly more time to achieve criteria for delayed-alternation testing than young adult rats (young adult: 16.3±1.9 sessions; aged: 24.1±2.5 sessions, p=0.044, FIG. 3A). Interestingly, once rats were fully habituated to the testing procedures and trained on the task, aged and young adult rats were not significantly different in overall working memory performance (young adult: 65.5±5.0% correct; aged: 69.7±1.5% correct, p=0.360, FIG. 3B) or in mean performance over the last 3 days of the characterization period, just prior to sacrifice (young adult: 71.0±5.8% correct; aged: 77.2±2.4% correct, p=0.303, FIG. 3B). Thus, the aged rats included in this experiment were considered cognitively unimpaired. The difference in pretreatment performance of aged rats included in the PKC inhibition experiment (section 3.2), and this group of aged rats illustrates the spectrum of cognitive abilities among the aged (Arnaiz, S. L., et al., Mol. Aspects. Med. (2004) 25: 91-101; Gage, F. H., et al., Neurobiol. Aging (1989) 10: 147-152; Olton, D. S. et al., Biomed. Environ. Sci. (1991) 4:166-172; West, R., et al., Brain Cogn. (2002) 49: 402-419).

### Cognitive Characterization: Prefrontal PKC Activation

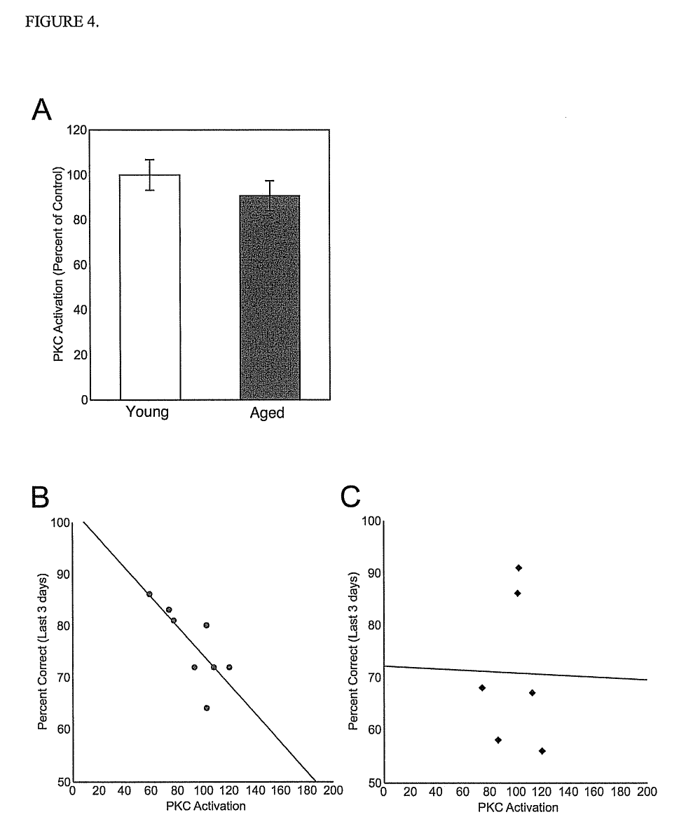

In an effort to understand whether there are underlying differences in PKC activity in the aged versus young adult prefrontal cortex, an ELISA assay was performed on prelimbic and infralimbic cortical regions taken from one hemisphere of cognitively characterized aged and young adult rats. PKC activity was assessed by measuring PKC levels in the membrane fraction, as PKC translocates to the plasma membrane when activated. Cytosolic measures of PKC were also obtained for each animal. Each sample was normalized to the mean activation detected in the young adult group (N=6). Independent t-tests did not indicate group differences in PKC activation (young adult: 100±6.8%; aged: 91±6.7%, p=0.364, FIG. 4A) or in cytosolic PKC concentration (young adult: 100±28.0%; aged: 78±13%, p=0.443, data not shown). These measures were performed by the Dr. Husseini Manji's lab at NIMH, who are experts in measuring PKC activity, at the request of Dr. Arnsten. Pearson's test revealed a significant inverse correlation between working memory performance and PKC activation in aged rats (r=−0.780, p=0.013, FIG. 4B). Performance was averaged over the last 3 days of the cognitive characterization, just prior to PKC activation measurements. Lower PKC activity levels predicted better cognitive performance in aged rats. No significant relationship between performance and PKC activation was detected among young adult rats (Pearson's test: r=−0.15, p=0.997, FIG. 4C). These findings support the hypothesis that, in the aged, basal PKC activation has an impairing effect on prefrontal mediated cognitive tasks.

### Example 5

### PKC Quantification

These assays presented in this example were conducted by the Manji lab at NIMH. Frozen medial prefrontal cortex was homogenized in 0.5 ml of ice-cold lysis buffer “A” (20 mM Tris, pH 7.5, 1 mM EGTA, 1 mM EDTA, 2.5 mM Na₄P₂O₇, 1 mM β-glycerophosphate, 1 mM dithiothreitol (DTT), 1% protease inhibitor cocktail, 1% phosphatase inhibitor cocktail I and II (Sigma-Aldrich, St. Louis, Mo.) by passing the tissue through a syringe with a 25G needle 15 times. Nuclei and debris were removed by centrifuging the homoginate at 1,000×g for 10 min. at 4° C. The homogenate was then centrifuged at 60,000×g for 1 h at 4° C. The resulting supernatant was used as cytosolic fraction, and the pellet was re-suspended in 0.5 ml of buffer “A” containing 0.15% Triton X-100, 150 mM NaCl and centrifuged again at the same settings. The resulting supernatant was used as the membrane fraction. The protein concentration of each condition was assessed by a Bradford assay and an appropriate volume of homogenization buffer was added to normalize the protein content between samples.

PKC activity was measured with Protein Kinase Assay kit (Calbiochem, San Diego, Calif.) according to the manufacturer's instructions with minor modifications. Because PKC is activated in its membrane bound state, PKC concentration in the membrane fraction was used as the measure of PKC activity. The reaction mixture (100 μl) contained: 10 μl sample, 25 mM Tris-HCl (pH 7.0), 0.5 mM EDTA, 1 mM EGTA, 5 mM β-mercaptoethanol, 2 mM CaCl₂, 3 mM MgCl₂, 50 μg/ml phosphatidylserine, and 0.1 mM ATP. The mixture was transferred to pseudosubstrate-coated 96-well plate and incubated at room temperature (RT) for 15 min. Stop solution (100 μl, 20% H₃PO₄) was added to the mixture to terminate the reaction. After washing the 96-well plate with PBS 5 times, 100 μl of biotinylated antibody was added to each well and incubated for 1 h at RT. Wells were washed with PBS between each incubation step. Peroxidase-conjugated streptavidin (100 μl) was added to each well and incubated for 1 h at RT. After a final wash, 100 μl of substrate solution (O-phenylenediamine and H₂O₂) was added to each well and allowed to incubate for 5 min. Stop solution (100 μl) was then added and optical density was measured at 492 nm in Victor 2 Multilabel Counter (Beckman Coulter, Inc., Fullerton, Calif.).

What is claimed is:

1. A method for improving cognition in an aged subject comprising providing to the subject an effective amount of a compound or salt of the structure: 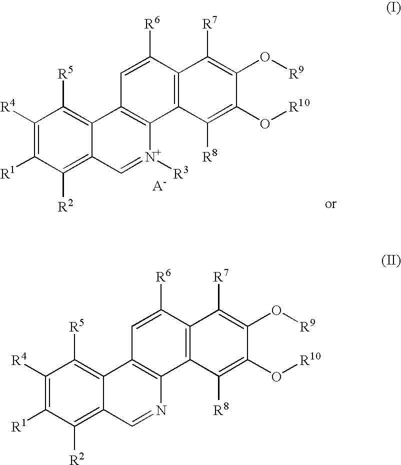
wherein:
R¹ and R² are independently selected from hydrogen, halogen, C₁-C₃alkyl, C₁-C₃alkoxy, or C₁-C₃alkylester;
R³ is hydrogen or C₁-C₆alkyl;
R⁴, R⁵, R⁶, R⁷ and R⁸ are independently selected from hydrogen, hydroxyl, C₁-C₃alkyl, —(CH₂)ₙO(C₁-C₃alkyl), —(CH₂)ₙO—C(═O)—(C₁-C₂)alkyl or —(CH₂)ₙC(═O)—O—(C₁-C₃)alkyl;
R⁹ and R¹⁰ are independently H, C₁-C₆ alkyl or R⁹ and R¹⁰ are taken together form a —(CH₂)ₘ— group to produce a 5-7 membered ring;
n is from 0 to 3;
m is from 1 to 3;
and A⁻ is an anion of a pharmaceutical salt, which forms a pharmaceutically acceptable salt with the quaternized amine group.

2. The method of claim 1 wherein R¹ and R² are both OCH₃ groups, R³ is a CH₃ group, R⁴, R⁵, R⁶, R⁷ and R⁸ are each hydrogen, R⁹ and R¹⁰ are each independently H or CH₃ or R⁹ and R¹⁰ are taken together to form a five-membered ring; and
A⁻ is Cl—, citrate, sulfate, or phosphate.

3. The method of claim 2, wherein R⁹ and R¹⁰ are taken together form a —CH₂— group to form a five-membered ring.

4. The method of claim 1, wherein the compound is chelerythrine.

5. The method of claim 1 or claim 4, wherein working memory is improved.

6. The method of claim 5, wherein the subject is a human subject of age 55 or greater.

7. The method of claim 6, wherein the subject is a human subject of age 70 or greater.

8. The method of claim 5, wherein about 0.1 micrograms/kg to about 10 micrograms/kg are administered to the subject per day.

9. The method of claim 5, wherein the compound is administered as a unit dosage form containing from about 3 micrograms to about 300 micrograms of the compound.

10. The method of claim 5, wherein the compound is administered non-centrally.

11. The method of claim 5, wherein the compound is administered orally.

12. The method of claim 1, wherein the effective amount is an amount effective to produce a significant improvement in a standard test of cognition.

13. The method of claim 5, wherein the effective amount is an amount effective to produce a significant improvement in a standard test of working memory.

14. The method of claim 1, wherein the method includes informing the subject that the compound can be used to improve cognition.

15. The method of claim 1, wherein the method includes informing the subject that compound can be used to improve working memory.
